(19) United States
(12) Patent Application Publication
(10) Pub. No.: US 2024/0262956 A1
(43) Pub. Date: Aug. 8, 2024

(54) ACCELERATOR FOR EPOXY RESINS

(71) Applicant: SIKA TECHNOLOGY AG, Baar (CH)

(72) Inventors: Edis KASEMI, Zürich (CH); Urs BURCKHARDT, Zürich (CH); Andreas KRAMER, Zürich (CH); Ursula STADELMANN, Zürich (CH)

(73) Assignee: SIKA TECHNOLOGY AG, Baar (CH)

(21) Appl. No.: 18/564,735

(22) PCT Filed: Jul. 4, 2022
(86) PCT No.: PCT/EP2022/068434

§ 371 Date: Nov. 28, 2023

(30) Foreign Application Priority Data
Jul. 12, 2021 (EP) 21185057.3

(51) Int. Cl. C08G 59/50; C07C 209/26; C07C 211/27; C09D 163/00; C09J 5/00; C09J 163/00

(52) U.S. Cl. CPC C08G 59/5033; C07C 209/26; C07C 211/27; C09D 163/00; C09J 5/00; C09J 163/00; C09J 2463/00

(57) ABSTRACT

An amine of the formula (I) is used for curing of epoxy resins and enables surprisingly rapid and undisrupted curing and high final hardnesses, with formation of nice, faultless surfaces with high gloss even in the case of two-dimensional application and low temperatures. It is suitable as curing agent, co-curing agent and/or incorporable accelerator for epoxy resin products such as, in particular, coatings, adhesives, potting compounds or shaped bodies.

### TECHNICAL FIELD

The invention relates to amines and to the use thereof for curing of epoxy resins.

### PRIOR ART

Epoxy resin compositions have various uses, including as adhesives or coatings in the building and manufacturing industries. It is advantageous when the compositions cure rapidly and reliably and hence can be subjected to mechanical load as early as possible after application. For faster curing, what are called accelerators are often used, especially in the case of low-temperature-curing systems. Substances from the category of the phenols in particular are known to be good accelerators. But many of the previously known accelerators, such as nonylphenol or tert-butylphenol, are barely usable any longer nowadays, they are toxic as such, are not incorporated into the polymer network on curing, and hence can lead to emissions, contamination or discoloration. The most effective accelerator for cold-curing systems is 2,4,6-tris(dimethylaminomethyl)phenol. However, it gives off a strong, unpleasant amine odor. Further known accelerators are acids such as salicylic acid in particular. Although it causes barely any odors, it results in a significant increase in viscosity, and the accelerating effect thereof is often inadequate.

Particularly advantageous accelerators are those that are incorporated into the polymer network on curing. The prior art discloses amine curing agents that additionally act as accelerators and are incorporated into the polymer network in the course of curing, for example 3-dimethylaminopropylamine (DMAPA), 3-(3-(dimethylamino)propylamino)propylamine (DMAPAPA) or 2,4,6-tris((2-dimethylaminopropyl)aminomethyl)phenol. But all three cause distinct blushing effects at low ambient temperature and in the case of two-dimensional application, and the accelerating effect thereof is still in need of improvement for many applications.

Alkylated amines are known as curing agents for epoxy resin coatings, for example from EP 3,180,383 or EP 3,344,677. They exhibit reliable curing at ambient temperatures and little tendency to blushing effects on two-dimensional application. But the curing rates achieved thereby are still in need of improvement.

### SUMMARY OF THE INVENTION

It is therefore an object of the present invention to provide a low-odor accelerator for curing epoxy resins, which enables particularly rapid curing, does not cause an increase in viscosity, and is incorporated into the cured polymer network. Surprisingly, this object is achieved by an amine of the formula (I) as described in claim 1. The amine of the formula (I) is preparable in a simple process by reductive alkylation of a dimethylamine with a benzonitrile that bears a formyl or acetyl group, and hydrogen. It has low odor and low viscosity, and shows excellent properties as curing agent and accelerator. It enables surprisingly rapid and undisrupted curing and high final hardnesses, with formation of nice, faultless surfaces with high gloss even in the case of two-dimensional application and low temperatures.

The amine of the formula (I) is suitable as curing agent, co-curing agent and/or incorporable accelerator for epoxy resin products such as, in particular, coatings, adhesives, potting compounds, shaped bodies or matrix resins for composite materials.

Further aspects of the invention are the subject of further independent claims. Particularly preferred embodiments of the invention are the subject of the dependent claims.

### Ways of Executing the Invention

The invention provides for the use of an amine of the formula (I) for curing of epoxy resins,

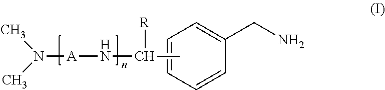

where n is 0 or 1 or 2, R is H or methyl and A is an alkylene radical having 2 to 6 carbon atoms.

Substance names beginning with “poly”, such as polyamine or polyepoxide, refer to substances that formally contain two or more of the functional groups that occur in their name per molecule.

A “primary amino group” refers to an amino group that is attached to a single organic radical and bears two hydrogen atoms; a “secondary amino group” refers to an amino group that is attached to two organic radicals that may also together be part of a ring and bears one hydrogen atom; and a “tertiary amino group” refers to an amino group that is attached to three organic radicals, two or three of which may also be part of one or more rings, and does not bear any hydrogen atom.

“Amine hydrogen” refers to the hydrogen atoms of primary and secondary amine groups.

“Amine hydrogen equivalent weight” refers to the mass of an amine or an amine-containing composition that contains one molar equivalent of amine hydrogen. It is expressed in units of “g/eq”.

The “epoxide equivalent weight” refers to the mass of an epoxy group-containing compound or composition that contains one molar equivalent of epoxy groups. It is expressed in units of “g/eq”.

A “thinner” refers to a substance that is soluble in an epoxy resin and lowers its viscosity, and that is not chemically incorporated into the epoxy resin polymer during the curing process.

“Molecular weight” refers to the molar mass (in grams per mole) of a molecule.

“Average molecular weight” refers to the number-average Mₙ of a polydisperse mixture of oligomeric or polymeric molecules, which is typically determined by gel-permeation chromatography (GPC) against polystyrene as standard.

“Pot life” refers to the maximum period of time from the mixing of the components and the application of an epoxy resin composition in which the mixed composition is in a sufficiently free-flowing state and has good ability to wet the substrate surfaces.

The “gel time” refers to the time interval from mixing the components of an epoxy resin composition until the gelation thereof.

“Room temperature” refers to a temperature of 23° C.

All industry standards and norms mentioned in the document refer to the versions valid at the date of first filing, unless stated otherwise.

Percent by weight (% by weight) values refer to the proportions by mass of a constituent in a composition based on the overall composition, unless stated otherwise. The terms “mass” and “weight” are used synonymously in the present document.

Preferably, R is H. These amines of the formula (I) enable particularly rapid curing.

Preferably, A is 1,2-ethylene, 1,2-propylene, 1,3-propylene, 1,4-butylene or 1,5-pentylene.

More preferably, A is 1,2-ethylene or 1,3-propylene, especially 1,3-propylene.

Preferably, n is 0 or 1. More preferably, n is 1.

Preferably, the amine of the formula (I) is selected from the group consisting of N,N-dimethyl-1,3-bis(aminomethyl)benzene, N-(2-dimethylaminoethyl)-1,3-bis(aminomethyl)benzene, N-(3-dimethylaminopropyl)-1,3-bis(aminomethyl)benzene, N,N-dimethyl-1,4-bis(aminomethyl)benzene, N-(2-dimethylaminoethyl)-1,4-bis(aminomethyl)benzene, N-(3-dimethylaminopropyl)-1,4-bis(aminomethyl)benzene, N,N-dimethyl-1,2-bis(aminomethyl)benzene, N-(2-dimethylaminoethyl)-1,2-bis(aminomethyl)benzene and N-(3-dimethylaminopropyl)-1,2-bis(aminomethyl)benzene.

The substituents on the benzene ring are preferably in 1,3 or in 1,4 positions, especially in 1,3 positions.

Among these, preference is given to N-(3-dimethylaminopropyl)-1,3-bis(aminomethyl)benzene or N-(3-dimethylaminopropyl)-1,4-bis(aminomethyl)benzene.

The amine of the formula (I) is preferably a constituent of a reaction product obtained from the reductive alkylation of at least one amine of the formula (II) with at least one benzonitrile of the formula (III) and hydrogen,

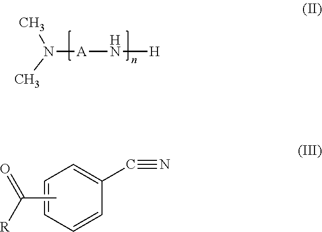

where n, R and A have the definitions already given.

A preferred amine of the formula (II) is dimethylamine, 2-dimethylaminoethylamine, 3-dimethylamino-2-propylamine, 3-dimethylaminopropylamine (DMAPA), 4-dimethylaminobutylamine, 5-dimethylaminopentylamine or 3-(3-(dimethylamino)propylamino)propylamine (DMAPAPA).

Particular preference is given to dimethylamine, 2-dimethylaminoethylamine or 3-dimethylaminopropylamine (DMAPA), especially DMAPA.

A suitable benzonitrile of the formula (III) is 2-formylbenzonitrile, 3-formylbenzonitrile or 4-formylbenzonitrile, 2-acetylbenzonitrile, 3-acetylbenzonitrile or 4-acetylbenzonitrile. Preference is given to 2-formylbenzonitrile, 3-formylbenzonitrile or 4-formylbenzonitrile, especially 3-formylbenzonitrile or 4-formylbenzonitrile, greatest preference to 3-formylbenzonitrile.

The amine of the formula (II) and the benzonitrile of the formula (III) are preferably used roughly stoichiometrically, especially in a molar ratio between the amine the formula (II) and the benzonitrile of the formula (III) of 0.9/1 to 1.1/1, preferably 0.9/1 to 1.0/1.

The reductive alkylation can be effected directly with molecular hydrogen, or indirectly by hydrogen or hydride transfer from other reagents such as formic acid or LiAIH₄. Preference is given to using molecular hydrogen.

The conditions in the reaction are advantageously chosen such that the nitrile group is likewise hydrogenated and the benzene ring is not hydrogenated.

Preference is given to conducting the reaction at a temperature of 40 to 120° C., and in the presence of a suitable catalyst. Preferred catalysts are palladium on charcoal (Pd/C), platinum on charcoal (Pt/C), Adams' catalyst or Raney nickel, especially palladium on charcoal or Raney nickel.

When molecular hydrogen is used, preference is given to working in a pressure apparatus at a hydrogen pressure of 5 to 150 bar, especially 10 to 120 bar.

The volatile components, especially any solvent present and water, are preferably removed from the reaction product after the reaction, especially by distillation or stripping.

The reaction product can be purified further, especially by distillation. This affords a reaction product having a particularly high content of amine of the formula (I).

The reaction product preferably contains a content of amine of the formula (I) of at least 70% by weight, more preferably at least 80% by weight, especially at least 90% by weight.

The reaction product may comprise the following compounds in particular as by-products:
compounds having imine and/or nitrile groups from the incomplete hydrogenation of the intermediates, in the case that n=1 for example of the formula

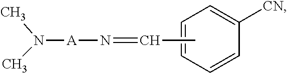

amines of the formula

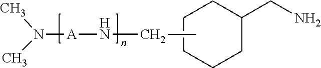

with a hydrogenated benzene ring,
amino alcohols of the formula

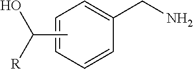

from the hydrogenation of unconverted benzonitrile of the formula (III),
polyalkylated amines, for example the following in the case of DMAPA and 3-formylbenzonitrile:

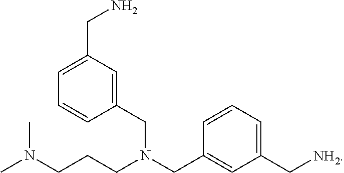 [SMILES: CN(C)CCCN(Cc1cccc(CN)c1)Cc1cccc(CN)c1]

The amine of the formula (I) is of low odor and low viscosity.

The amine of formula (I), especially in the form of the reaction product described, is used for curing of epoxy resins.

Suitable epoxy resins are especially glycidyl ethers of commercial mono- or polyfunctional alcohols or phenols, especially the epoxy resins specified in this document.

Preference is given to mixing the amine of the formula (I), the epoxy resin and any further substances with one another, where the ratio of the groups reactive toward the epoxy groups, especially the amine hydrogens of the amine of the formula (I), and the epoxy groups is preferably in the range from 0.5 to 1.5, especially 0.7 to 1.2.

The curing can be effected at ambient temperature, especially in the range from 5 to 40° C., or at elevated temperature, especially in the range from 40 to 150° C., preferably 50 to 120° C.

The amine of the formula (I) enables surprisingly rapid and undisrupted curing and high final hardnesses, with formation of nice, faultless surfaces with high gloss even in the case of two-dimensional application and low temperatures.

The amine of the formula (I) can be used in combination with further curing agents.

The amine of the formula (I) may be used in partly adducted form with at least one epoxy resin or monoepoxide, especially with an epoxy resin or monoepoxide having an average epoxy equivalent weight in the range from 150 to 500 g/eq, preferably 156 to 250 g/eq.

Preference is given to amine-functional adducts from the reaction of at least one amine of the formula (I) and at least one epoxy resin in a stoichiometric ratio in the range from 1 to 10, preferably 1 to 5, mol of amine of the formula (I) per molar equivalent of epoxy groups.

The invention further provides a curing agent for epoxy resins comprising at least one amine of the formula (I) and at least one further constituent selected from further amines A1, accelerators and thinners.

The further amine A1 is preferably not an amine of the formula (I).

The curing agent preferably contains 1% to 99% by weight, more preferably 2% to 90% by weight, more preferably 3% to 75% by weight, especially preferably 5% to 50% by weight, in particular 7% to 30% by weight, of amine of the formula (I), based on the overall curing agent.

The curing agent is preferably not water-based. It preferably contains less than 15% by weight, especially less than 10% by weight, of water, based on the overall curing agent. Such a curing agent is particularly suitable for nonaqueous epoxy resin products.

Suitable further amines are especially amines having at least two, preferably at least three, amine hydrogens that do not conform to the formula (I).

The curing agent preferably comprises at least one further amine A1 having at least three aliphatic amine hydrogens.

The following are especially suitable for this purpose: N-benzylethane-1,2-diamine, N-benzylpropane-1,2-diamine, N-benzyl-1,3-bis(aminomethyl)benzene, N-(2-phenylethyl)-1,3-bis(aminomethyl)benzene, N-(2-ethylhexyl)-1,3-bis(aminomethyl)benzene, 2,2-dimethylpropane-1,3-diamine, pentane-1,3-diamine (DAMP), pentane-1,5-diamine, 1,5-diamino-2-methylpentane (MPMD), 2-butyl-2-ethylpentane-1,5-diamine (C11-neodiamine), hexane-1,6-diamine, 2,5-dimethylhexane-1,6-diamine, 2,2(4),4-trimethylhexane-1,6-diamine (TMD), heptane-1,7-diamine, octane-1,8-diamine, nonane-1,9-diamine, decane-1,10-diamine, undecane-1,11-diamine, dodecane-1,12-diamine, 1,2-, 1,3- or 1,4-diaminocyclohexane, 1,3-bis(aminomethyl)cyclohexane, 1,4-bis(aminomethyl)cyclohexane, bis(4-aminocyclohexyl)methane, bis(4-amino-3-methylcyclohexyl)methane, bis(4-amino-3-ethylcyclohexyl)methane, bis(4-amino-3,5-dimethylcyclohexyl)methane, bis(4-amino-3-ethyl-5-methylcyclohexyl)methane, 1-amino-3-aminomethyl-3,5,5-trimethylcyclohexane (IPDA), 2(4)-methyl-1,3-diaminocyclohexane, 2,5(2,6)-bis(aminomethyl)bicyclo[2.2.1]heptane (NBDA), 3(4),8(9)-bis(aminomethyl)tricyclo[5.2.1.0^{2.6}]decane, 1,4-diamino-2,2,6-trimethylcyclohexane (TMCDA), menthane-1,8-diamine, 3,9-bis(3-aminopropyl)-2,4,8,10-tetraoxaspiro[5.5]undecane, 1,3-bis(aminomethyl)benzene (MXDA), 1,4-bis(aminomethyl)benzene, bis(2-aminoethyl) ether, 3,6-dioxaoctane-1,8-diamine, 4,7-dioxadecane-1,10-diamine, 4,7-dioxadecane-2,9-diamine, 4,9-dioxadodecane-1,12-diamine, 5,8-dioxadodecane-3,10-diamine, 4,7,10-trioxatridecane-1,13-diamine or higher oligomers of these diamines, bis(3-aminopropyl)polytetrahydrofurans or other polytetrahydrofurandiamines, polyoxyalkylenediamines or -triamines, especially polyoxypropylenediamines or polyoxypropylenetriamines such as, in particular, Jeffamine® D-230, Jeffamine® D-400 or Jeffamine® T-403 (all from Huntsman), polyalkyleneamines such as diethylenetriamine (DETA), triethylenetetramine (TETA), tetraethylenepentamine (TEPA), pentaethylenehexamine (PEHA), dipropylenetriamine (DPTA), N-(2-aminoethyl)propane-1,3-diamine (N3 amine), N,N′-bis(3-aminopropyl)ethylenediamine (N4 amine), N,N′-bis(3-aminopropyl)-1,4-diaminobutane, N5-(3-aminopropyl)-2-methylpentane-1,5-diamine, N3-(3-aminopentyl)pentane-1,3-diamine, N5-(3-amino-1-ethylpropyl)-2-methylpentane-1,5-diamine, N,N′-bis(3-amino-1-ethylpropyl)-2-methylpentane-1,5-diamine, 3-(2-aminoethyl)aminopropylamine or bis(hexamethylene)triamine (BHMT), N-benzylated polyalkyleneamines having one or two benzyl groups, especially benzylated DETA, TETA, N3 amine, N4 amine or DPTA, N-aminoethylpiperazine, 3-(3-(dimethylamino)propylamino)propylamine (DMAPAPA), amine-functional adducts of the amines mentioned with epoxides, or mixtures of two or more of these amines.

The curing agent preferably comprises at least one further amine A1 selected from the group consisting of N-benzylethane-1,2-diamine, MPMD, TMD, 1,2-diaminocyclohexane, 1,3-bis(aminomethyl)cyclohexane, 1,4-bis(aminomethyl)cyclohexane, bis(4-aminocyclohexyl)methane, IPDA, 2(4)-methyl-1,3-diaminocyclohexane, NBDA, MXDA, DETA, TETA, TEPA, N3 amine, N4 amine, DPTA, BHMT, DMAPAPA, polyoxypropylene diamines having an average molecular weight Mₙ in the range from 200 to 500 g/mol, and polyoxypropylene triamines having an average molecular weight Mₙ in the range from 300 to 500 g/mol.

Among these, preference is given to N-benzylethane-1,2-diamine. This enables coatings having particularly nice surfaces.

Among these, preference is also given to MXDA, 1,3-bis(aminomethyl)cyclohexane or 1,4-bis(aminomethyl)cyclohexane. These permit particularly rapid curing.

Among these, preference is also given to IPDA. This enables an inexpensive curing agent with high glass transition temperature.

Among these, preference is also given to DETA, TETA, TEPA or N4 amine. These enable inexpensive adhesives having particularly high bonding power.

The curing agent may especially comprise more than one further amine A1.

Suitable accelerators are especially acids or compounds hydrolyzable to acids, especially organic carboxylic acids such as acetic acid, benzoic acid, salicylic acid, 2-nitrobenzoic acid, lactic acid, organic sulfonic acids such as methanesulfonic acid, p-toluenesulfonic acid or 4-dodecylbenzenesulfonic acid, sulfonic esters, other organic or inorganic acids, such as phosphoric acid in particular, or mixtures of the abovementioned acids and acid esters; nitrates such as calcium nitrate in particular; tertiary amines such as, in particular, 1,4-diazabicyclo[2.2.2]octane, benzyldimethylamine, α-methylbenzyldimethylamine, triethanolamine, dimethylaminopropylamine, imidazoles such as, in particular, N-methylimidazole, N-vinylimidazole or 1,2-dimethylimidazole, salts of such tertiary amines, quaternary ammonium salts, such as benzyltrimethylammonium chloride in particular, amidines, such as 1,8-diazabicyclo[5.4.0]undec-7-ene in particular, guanidines, such as 1,1,3,3-tetramethylguanidine in particular, phenols, especially bisphenols, phenolic resins or Mannich bases such as, in particular, 2-(dimethylaminomethyl)phenol, 2,4,6-tris(dimethylaminomethyl)phenol or polymers produced from phenol, formaldehyde and N,N-dimethylpropane-1,3-diamine, phosphites such as, in particular, di- or triphenyl phosphites, or compounds having mercapto groups.

Preference is given to acids, nitrates, tertiary amines or Mannich bases, especially salicylic acid, calcium nitrate or 2,4,6-tris(dimethylaminomethyl)phenol, or a combination of these accelerators.

Suitable thinners are especially n-propanol, isopropanol, n-butanol, isobutanol, tert-butanol, 1-pentanol, 2-pentanol, 3-pentanol, 2-methyl-1-butanol, 3-methyl-1-butanol, 3-methyl-2-butanol, n-hexanol, 2-ethylhexanol, xylene, 2-methoxyethanol, dimethoxyethanol, 2-ethoxyethanol, 2-propoxyethanol, 2-isopropoxyethanol, 2-butoxyethanol, 2-phenoxyethanol, 2-benzyloxyethanol, benzyl alcohol, ethylene glycol, ethylene glycol dimethyl ether, ethylene glycol diethyl ether, ethylene glycol dibutyl ether, ethylene glycol diphenyl ether, diethylene glycol, diethylene glycol monomethyl ether, diethylene glycol monoethyl ether, diethylene glycol mono-n-butyl ether, diethylene glycol dimethyl ether, diethylene glycol diethyl ether, diethylene glycol di-n-butyl ether, propylene glycol butyl ether, propylene glycol phenyl ether, dipropylene glycol, dipropylene glycol monomethyl ether, dipropylene glycol dimethyl ether, dipropylene glycol di-n-butyl ether, 2,2,4-trimethylpentane-1,3-diol monoisobutyrate, diphenylmethane, diisopropylnaphthalene, mineral oil fractions, for example Solvesso® grades (from Exxon), alkylphenols such as tert-butylphenol, nonylphenol, dodecylphenol, cardanol, styrenated phenol, bisphenols, aromatic hydrocarbon resins, especially types containing phenol groups, alkoxylated phenol, especially ethoxylated or propoxylated phenol, especially 2-phenoxyethanol, adipates, sebacates, phthalates, benzoates, organic phosphoric or sulfonic esters or sulfonamides.

Preference among these is given to thinners having a boiling point of more than 200° C., especially benzyl alcohol, styrenated phenol, ethoxylated phenol, aromatic hydrocarbon resins containing phenol groups, such as, in particular, the Novares® grades LS 500, LX 200, LA 300 or LA 700 (from Rutgers), diisopropylnaphthalene or cardanol, especially benzyl alcohol.

Thinners containing phenol groups are effective also as accelerators.

The curing agent may comprise further constituents, especially:
further adducts, especially adducts of MPMD or ethane-1,2-diamine or propane-1,2-diamine with cresyl glycidyl ether or aromatic epoxy resins, in which unreacted MPMD, ethane-1,2-diamine or propane-1,2-diamine has been removed by distillation after the reaction,
monoamines such as, in particular, benzylamine or furfurylamine,
polyamidoamines, especially reaction products of a mono- or polybasic carboxylic acid, or the ester or anhydride thereof, especially a dimer fatty acid, with a polyamine used in stoichiometric excess, especially DETA or TETA,
Mannich bases, especially phenalkamines, i.e. reaction products of phenols, especially cardanol, with aldehydes, especially formaldehyde, and polyamines,
aromatic polyamines such as in particular 4,4′-, 2,4′ and/or 2,2′-diaminodiphenylmethane, 2,4(6)-toluenediamine, 3,5-dimethylthio-2,4(6)-toluenediamine or 3,5-diethyl-2,4(6)-tolylenediamine,
compounds having mercapto groups, especially liquid mercaptan-terminated polysulfide polymers, mercaptan-terminated polyoxyalkylene ethers, mercaptan-terminated polyoxyalkylene derivatives, polyesters of thiocarboxylic acids, 2,4,6-trimercapto-1,3,5-triazine, triethylene glycol dimercaptan or ethanedithiol,
surface-active additives, especially defoamers, deaerating agents, wetting agents, dispersants or leveling agents, or
stabilizers, especially stabilizers against oxidation, heat, light or UV radiation.

The invention further provides an epoxy resin composition comprising
a resin component comprising at least one epoxy resin and
a curing agent component comprising at least one amine of the formula (I) or a curing agent comprising at least one amine of the formula (I), as described above.

A suitable epoxy resin is obtained in a known manner especially from the reaction of epichlorohydrin with polyols, polyphenols or amines.

Suitable epoxy resins are especially aromatic epoxy resins, especially the glycidyl ethers of:
bisphenol A, bisphenol F or bisphenol A/F, where A stands for acetone and F for formaldehyde used as reactants in the production of these bisphenols. In the case of bisphenol F, positional isomers may also be present, more particularly ones derived from 2,4′- or 2,2′-hydroxyphenylmethane.
dihydroxybenzene derivatives such as resorcinol, hydroquinone or catechol;
further bisphenols or polyphenols such as bis(4-hydroxy-3-methylphenyl)methane, 2,2-bis(4-hydroxy-3-methylphenyl)propane (bisphenol C), bis(3,5-dimethyl-4-hydroxyphenyl)methane, 2,2-bis(3,5-dimethyl-4-hydroxyphenyl)propane, 2,2-bis(3,5-dibromo-4-hydroxyphenyl)propane, 2,2-bis(4-hydroxy-3-tert-butylphenyl)propane, 2,2-bis(4-hydroxyphenyl)butane (bisphenol B), 3,3-bis(4-hydroxyphenyl)pentane, 3,4-bis(4-hydroxyphenyl)hexane, 4,4-bis(4-hydroxyphenyl)heptane, 2,4-bis(4-hydroxyphenyl)-2-methylbutane, 2,4-bis(3,5-dimethyl-4-hydroxyphenyl)-2-methylbutane, 1,1-bis(4-hydroxyphenyl)cyclohexane (bisphenol Z), 1,1-bis(4-hydroxyphenyl)-3,3,5-trimethylcyclohexane (bisphenol TMC), 1,1-bis(4-hydroxyphenyl)-1-phenylethane, 1,4-bis[2-(4-hydroxyphenyl)-2-propyl]benzene (bisphenol P), 1,3-bis[2-(4-hydroxyphenyl)-2-propyl]benzene (bisphenol M), 4,4′-dihydroxydiphenyl (DOD), 4,4′-dihydroxybenzophenone, bis(2-hydroxynaphth-1-yl)methane, bis(4-hydroxynaphth-1-yl)methane, 1,5-dihydroxynaphthalene, tris(4-hydroxyphenyl)methane, 1,1,2,2-tetrakis(4-hydroxyphenyl)ethane, bis(4-hydroxyphenyl) ether or bis(4-hydroxyphenyl) sulfone;
novolaks, which are especially condensation products of phenol or cresols with formaldehyde or paraformaldehyde or acetaldehyde or crotonaldehyde or isobutyraldehyde or 2-ethylhexanal or benzaldehyde or furfural;
aromatic amines such as aniline, toluidine, 4-aminophenol, 4,4′-methylenediphenyldiamine, 4,4′-methylenediphenyldi(N-methyl)amine, 4,4′-[1,4-phenylenebis(1-methylethylidene)]bisaniline (bisaniline P) or 4,4′-[1,3-phenylenebis(1-methylethylidene)]bisaniline (bisaniline M).

Further suitable epoxy resins are aliphatic or cycloaliphatic polyepoxides, especially
glycidyl ethers of saturated or unsaturated, branched or unbranched, cyclic or open-chain di-, tri- or tetrafunctional C₂ to C₃₀ alcohols, especially ethylene glycol, propylene glycol, butylene glycol, hexanediol, octanediol, polypropylene glycols, dimethylolcyclohexane, neopentyl glycol, dibromoneopentyl glycol, castor oil, trimethylolpropane, trimethylolethane, pentaerythritol, sorbitol or glycerol, or alkoxylated glycerol or alkoxylated trimethylolpropane;
a hydrogenated bisphenol A, F or A/F liquid resin or the glycidylation products of hydrogenated bisphenol A, F or A/F;
an N-glycidyl derivative of amides or heterocyclic nitrogen bases, such as triglycidyl cyanurate or triglycidyl isocyanurate, or reaction products of epichlorohydrin with hydantoin.

The epoxy resin is preferably a liquid resin or a mixture comprising two or more liquid epoxy resins.

“Liquid epoxy resin” refers to an industrial polyepoxide having a glass transition temperature below 25° C.

The resin component optionally additionally contains proportions of solid epoxy resin.

The epoxy resin is especially a liquid resin based on a bisphenol or novolaks, especially one having an average epoxy equivalent weight in the range from 156 to 210 g/eq.

A bisphenol A diglycidyl ether and/or bisphenol F diglycidyl ether, such as those commercially available from Olin, Huntsman or Momentive, is particularly suitable. These liquid resins have low viscosity for epoxy resins and permit rapid curing and high hardnesses. They may contain proportions of solid bisphenol A resin or novolak epoxy resins.

Also particularly suitable are phenol-formaldehyde novolak glycidyl ethers, especially having an average functionality in the range from 2.3 to 4, preferably 2.5 to 3. They may contain proportions of other epoxy resins, especially bisphenol A diglycidyl ether or bisphenol F diglycidyl ether.

The resin component may comprise a reactive diluent.

Preferred reactive diluents are reactive diluents containing epoxy groups, especially butanediol diglycidyl ether, hexanediol diglycidyl ether, trimethylolpropane di- or triglycidyl ether, phenyl glycidyl ether, cresyl glycidyl ether, guaiacol glycidyl ether, 4-methoxyphenyl glycidyl ether, p-n-butylphenyl glycidyl ether, p-tert-butylphenyl glycidyl ether, 4-nonylphenyl glycidyl ether, 4-dodecylphenyl glycidyl ether, cardanol glycidyl ether, benzyl glycidyl ether, allyl glycidyl ether, butyl glycidyl ether, hexyl glycidyl ether, 2-ethylhexyl glycidyl ether, or glycidyl ethers of natural alcohols, such as, in particular, C₈ to C₁₀ or C₁₂ to C₁₄ or C₁₃ to C₁₅ alkyl glycidyl ethers.

The epoxy resin composition preferably comprises at least one further constituent selected from the group consisting of thinners, accelerators, stabilizers, surface-active additives, fillers and pigments.

Suitable fillers, accelerators, stabilizers and surface-active additives are especially those already mentioned.

Suitable fillers are, in particular, ground or precipitated calcium carbonate, which is optionally coated with fatty acid, especially stearates, baryte (heavy spar), talc, quartz powder, quartz sand, silicon carbide, iron mica, dolomite, wollastonite, kaolin, mica (potassium aluminum silicate), molecular sieves, aluminum oxide, zinc oxide, aluminum-doped zinc oxide, aluminum hydroxide, magnesium hydroxide, silica, cement, gypsum, fly ash, carbon black, graphite, groundwood, metal powders such as aluminum, copper, iron, zinc, silver or steel, PVC powder or hollow beads. Preference among these is given to calcium carbonate, baryte, quartz powder, talc, or a combination thereof.

Suitable pigments are especially titanium dioxides, iron oxides, chromium(III) oxides, anticorrosion pigments, organic pigments or carbon black, especially titanium dioxides.

The epoxy resin composition may optionally comprise further auxiliaries and additives, especially the following:
reactive diluents, especially those already mentioned, or epoxidized soybean oil or linseed oil, compounds containing acetoacetate groups, especially acetoacetylated polyols, butyrolactone, carbonates, aldehydes, isocyanates or silicones having reactive groups;
polymers, especially polyamides, polysulfides, polyvinyl formal (PVF), polyvinyl butyral (PVB), polyurethanes (PUR), polymers having carboxyl groups, polyamides, butadiene-acrylonitrile copolymers, styrene-acrylonitrile copolymers, butadiene-styrene copolymers, homo- or copolymers of unsaturated monomers, especially from the group comprising ethylene, propylene, butylene, isobutylene, isoprene, vinyl acetate or alkyl (meth)acrylates, especially chlorosulfonated polyethylenes or fluorine-containing polymers or sulfonamide-modified melamines;
fibers, especially glass fibers, carbon fibers, cellulose fibers, metal fibers, ceramic fibers or polymer fibers such as polyamide fibers or polyethylene fibers;
further solids, especially wood pellets or cellulose;
nanofillers, especially carbon nanotubes;
rheology modifiers, especially thickeners or antisettling agents;
adhesion improvers, especially organoalkoxysilanes;
flame-retardant substances, especially the aluminum hydroxide or magnesium hydroxide fillers already mentioned, antimony trioxide, antimony pentoxide, boric acid (B(OH)₃), zinc borate, zinc phosphate, melamine borate, melamine cyanurate, ammonium polyphosphate, melamine phosphate, melamine pyrophosphate, polybrominated diphenyl oxides or diphenyl ethers, phosphates such as, in particular, diphenyl cresyl phosphate, resorcinol bis(diphenyl phosphate), resorcinol diphosphate oligomer, tetraphenylresorcinol diphosphite, ethylenediamine diphosphate, bisphenol A bis(diphenyl phosphate), tris(chloroethyl) phosphate, tris(chloropropyl) phosphate, tris(dichloroisopropyl) phosphate, tris[3-bromo-2,2-bis(bromomethyl)propyl] phosphate, tetrabromobisphenol A, bis(2,3-dibromopropyl ether) of bisphenol A, brominated epoxy resins, ethylenebis(tetrabromophthalimide), ethylenebis(dibromonorbornanedicarboximide), 1,2-bis(tribromophenoxy)ethane, tris(2,3-dibromopropyl) isocyanurate, tribromophenol, hexabromocyclododecane, bis(hexachlorocyclopentadieno)cyclooctane or chloroparaffins; or
further additives, especially dispersed paraffin wax or biocides.

The epoxy resin composition preferably has only a low content of thinners. It preferably contains less than 30% by weight, more preferably less than 20% by weight, in particular less than 10% by weight, of thinner, based on the overall epoxy resin composition.

The epoxy resin composition preferably has only a low content of water, preferably less than 5% by weight, especially less than 1% by weight, of water, based on the overall epoxy resin composition.

The resin component and the curing agent component of the epoxy resin composition are stored in separate containers. Further constituents of the epoxy resin composition may be present as a constituent of the resin component or of the curing agent component; further constituents reactive toward epoxy groups are preferably a constituent of the curing agent component. It is likewise possible for further constituents to be present as a separate, further component.

A suitable container for storage of the resin component or the curing agent component is especially a vat, a hobbock, a bag, a bucket, a can, a cartridge or a tube. The components are storable, meaning that they can be stored prior to use for several months up to one year or longer without any change in their respective properties to a degree relevant to their use.

The resin component and curing agent component are mixed shortly before or during application. The mixing ratio is preferably chosen such that the molar ratio of the groups reactive toward epoxy groups relative to the epoxy groups is in the range from 0.5 to 1.5, especially 0.7 to 1.2. In parts by weight, the mixing ratio between the resin component and the curing agent component is typically within a range from 1:2 to 20:1.

The components are mixed continuously or in batches by means of a suitable method, taking care to ensure that not too much time elapses between the mixing of the components and the application, and that application takes place within the pot life. Mixing and application can be effected at ambient temperature, which is typically in the range from about 5 to 40° C., preferably about 10 to 35° C., or at elevated temperature, especially in the range from 40 to 150° C., preferably 50 to 120° C.

Upon mixing the components, the curing of the epoxy resin composition by chemical reaction commences. Primary and secondary amino groups, and any further groups present that are reactive toward epoxy groups, react with the epoxy groups, resulting in ring opening thereof. As a result primarily of these reactions, the composition polymerizes and thereby cures.

Curing typically extends over a few hours to days. The duration depends on factors including the temperature, the reactivity of the constituents, the stoichiometry thereof, and the presence/amount of accelerators. The amine of the formula (I) enables particularly rapid curing, especially also at low temperatures.

When freshly mixed, the epoxy resin composition has low viscosity. The viscosity at 20° C. 5 minutes after the resin component and curing agent component have been mixed is preferably in the range from 0.2 to 20 mPa·s, preferably 0.3 to 10 mPa·s, especially 0.3 to 5 mPa·s, measured using a cone-plate viscometer at a shear rate of 10 s⁻¹.

The epoxy resin composition is applied to at least one substrate and/or to at least one casting mold.

Suitable substrates are especially:
glass, glass ceramic, concrete, mortar, cement screed, fiber cement, brick, tile, plaster or natural rocks such as granite or marble;
repair compounds or leveling compounds based on PCC (polymer-modified cement mortar) or ECC (epoxy resin-modified cement mortar);
metals or alloys such as aluminum, iron, steel, copper, other nonferrous metals, including surface-finished metals or alloys such as galvanized or chrome-plated metals;
asphalt or bitumen;
leather, textiles, paper, wood, wood-based materials bonded with resins, for example phenolic, melamine or epoxy resins, resin-textile composites or further polymer composites;
plastics, such as rigid and flexible PVC, polycarbonate, polystyrene, polyester, polyamide, PMMA, ABS, SAN, epoxy resins, phenolic resins, PUR, POM, TPO, PE, PP, EPM or EPDM, in each case untreated or surface-treated, for example by means of plasma, corona or flames;
fiber-reinforced plastics, such as carbon fiber-reinforced plastics (CFRP), glass fiber-reinforced plastics (GFRP), and sheet molding compounds (SMC);
insulation foams, especially made of EPS, XPS, PUR, PIR, rock wool, glass wool or foamed glass;
coated or painted substrates, especially painted tiles, coated concrete, powder-coated metals or alloys or painted metal sheets;
coatings, paints or varnishes, especially coated floors that have been overcoated with a further floor covering layer.

The substrates can if required be pretreated prior to application, especially by physical and/or chemical cleaning methods or the application of an activator or a primer.

The substrates are especially coated and/or adhesively bonded.

A suitable casting mold is an apparatus into which the mixed, liquid epoxy resin composition is poured and in which it is cured, and from which it is or can be demolded after curing, where the cured composition forms a shaped body.

The invention further provides a cured composition obtained from the epoxy resin composition described after mixing of the resin component and the curing agent component.

The epoxy resin composition is preferably used as coating, primer, adhesive, sealant, potting compound, casting resin, impregnating resin, or as shaped body or matrix for composite materials such as, in particular, CFRP (containing carbon fibers) or GFRP (containing glass fibers) or wood composites.

The invention further provides a method of coating, comprising the steps of
(i) applying the mixed epoxy resin composition to at least one substrate,
(ii) optionally applying a further layer of the mixed epoxy resin coating to the cured layer.

Suitable substrates are those already mentioned.

Preference is given here to using the epoxy resin composition as a primer, in which case the substrate is especially concrete, mortar, cement screed, or a metal, especially steel. Such an epoxy resin composition is preferably largely free of fillers and serves to strengthen the substrate, to close any pores, and to ensure good adhesion between the substrate and further layers.

Further preferably, the epoxy resin composition is used as a floor coating. Such an epoxy resin composition preferably comprises fillers and pigments and is applied two-dimensionally in one or more operations overall, especially in a layer thickness of 0.1 to 5 mm, preferably as a self-leveling coating, and left to cure. The substrate is preferably an optionally primer-pretreated concrete, mortar, cement screed.

Further preferably, the epoxy resin coating is used as a protective coating, especially as an anticorrosion coating on steel, or as a topcoat on floor coatings. Such a coating is preferably applied in a spraying method, especially in a layer thickness of 0.05 to 0.5 mm.

The invention further provides a method of bonding, comprising the applying of the mixed epoxy resin composition
either to at least one of the substrates to be bonded and joining the substrates to form a bond,
or into a cavity or gap between two or more substrates and optionally inserting an anchor into the cavity or gap.

An “anchor” refers here more particularly to a rebar, a threaded rod or a bolt. An anchor is in particular thus adhesive-bonded or anchored in a wall, ceiling or foundation in such a way that a portion thereof is bonded in a force-fitting manner and a portion thereof protrudes and can be subjected to a construction load.

Identical or different substrates may be bonded.

The invention further provides a method of producing a shaped body, comprising the introducing of the mixed epoxy resin composition into a casting mold, followed by the curing of the mixed composition, optionally under pressure and optionally by means of supplied heat.

The epoxy resin composition, before being cured, may be admixed with fibers, powders or pellets, especially with carbon fibers, glass fibers or wood powder and/or pellets, in order to obtain a composite material.

The shaped body may be produced in one shot in the casting mold, or it may be built up in layers.

After curing, the shaped body is preferably removed from the casting mold. It can be processed further, especially by cutting, drilling, drawing, grinding or polishing.

The use of an amine of the formula (I) for curing of epoxy resins, or a method of coating, bonding or producing a shaped body, gives rise to an article.

The article is preferably a built structure or part thereof, especially a built structure above or below ground, an office, an industrial hall, a sports hall, a cold room, a silo, a bridge, a roof, a staircase, a floor, a balcony, a terrace or a parking deck, or an industrial good or a consumer good, especially a lamella, a panel, a pier, an offshore platform, a lock gate, a crane, a bulkhead, a pipeline, a container or a rotor blade of a wind turbine, or a mode of transport such as in particular an automobile, a truck, a rail vehicle, a ship, an aircraft or helicopter, or an installable component thereof.

The amine of the formula (I) is preferably used as curing agent, co-curing agent and/or incorporable accelerator.

The invention further provides an amine of the formula (Ia)

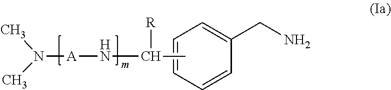

where m is 1 or 2, R is H or methyl and A is an alkylene radical having 2 to 6 carbon atoms.

Preferably, m is 1.

Preferably, R is H.

Preferably, A is 1,2-ethylene, 1,2-propylene, 1,3-propylene, 1,4-butylene or 1,5-pentylene, more preferably 1,2-ethylene or 1,3-propylene, especially 1,3-propylene.

Preferably, the amine of the formula (Ia) is selected from the group consisting of N-(2-dimethylaminoethyl)-1,3-bis(aminomethyl)benzene, N-(3-dimethylaminopropyl)-1,3-bis(aminomethyl)benzene, N-(2-dimethylaminoethyl)-1,4-bis(aminomethyl)benzene, N-(3-dimethylaminopropyl)-1,4-bis(aminomethyl)benzene, N-(2-dimethylaminoethyl)-1,2-bis(aminomethyl)benzene and N-(3-dimethylaminopropyl)-1,2-bis(aminomethyl)benzene.

The substituents on the benzene ring are preferably in 1,3 or in 1,4 positions.

More preferably, the amine of the formula (Ia) is N-(3-dimethylaminopropyl)-1,3-bis(aminomethyl)benzene or N-(3-dimethylaminopropyl)-1,4-bis(aminomethyl)benzene.

The invention further provides a reaction product comprising at least one amine of the formula (Ia), obtained from the reductive alkylation of at least one amine of the formula (IIa) with at least one benzonitrile of the formula (III) and hydrogen,

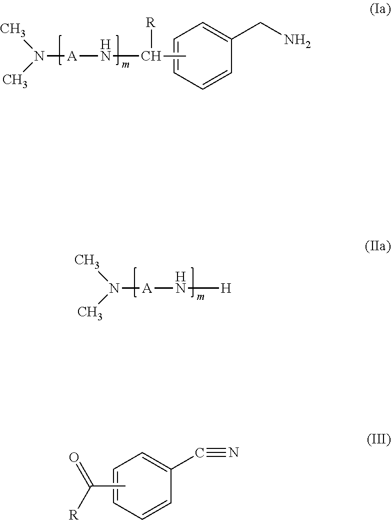

where m, R and A have the definitions already given.

A preferred amine of the formula (IIa) is 2-dimethylaminoethylamine, 3-dimethylamino-2-propylamine, 3-dimethylaminopropylamine (DMAPA), 4-dimethylaminobutylamine, 5-dimethylaminopentylamine or 3-(3-(dimethylamino)propylamino)propylamine (DMAPAPA).

Particular preference is given to 2-dimethylaminoethylamine DMAPA, especially DMAPA.

A suitable benzonitrile of the formula (III) is 2-formylbenzonitrile, 3-formylbenzonitrile or 4-formylbenzonitrile, 2-acetylbenzonitrile, 3-acetylbenzonitrile or 4-acetylbenzonitrile. Preference is given to 2-formylbenzonitrile, 3-formylbenzonitrile or 4-formylbenzonitrile, especially 3-formylbenzonitrile or 4-formylbenzonitrile, greatest preference to 3-formylbenzonitrile.

The amine of the formula (II) and the benzonitrile of the formula (III) are preferably used roughly stoichiometrically, especially in a molar ratio between the amine the formula (II) and the benzonitrile of the formula (III) of 0.9/1 to 1.1/1, preferably 0.9/1 to 1.0/1.

The reductive alkylation is conducted as described above.

The reaction product can be purified further, especially by distillation. This affords a reaction product having a particularly high content of amine of the formula (Ia).

The reaction product preferably contains a content of amine of the formula (Ia) of at least 70% by weight, more preferably at least 80% by weight, especially at least 90% by weight.

The amine of the formula (Ia) is of low odor and low viscosity.

The amine of formula (Ia), especially in the form of the reaction product described, is preferably used for curing of epoxy resins.

### EXAMPLES

Working examples are adduced hereinafter, which are intended to further elucidate the invention described. The invention is of course not limited to these described working examples.

“AHEW” stands for amine hydrogen equivalent weight.

“EEW” stands for epoxy equivalent weight.

“Standard climatic conditions” (“SCC”) refer to a temperature of 23±1° C. and a relative humidity of 50±5%.

The chemicals used were unless otherwise stated from Sigma-Aldrich Chemie GmbH.

### Description of the Measurement Methods

Viscosity was measured on a thermostated Rheotec RC30 cone-plate viscometer (cone diameter 50 mm, cone angle 10, cone tip-plate distance 0.05 mm). Low-viscosity samples having a viscosity of less than 100 mPa·s were measured at a shear rate of 100 s⁻¹, higher-viscosity samples at a rate of 10 s⁻¹.

Amine value was determined by titration (with 0.1 N HCIO₄ in acetic acid against crystal violet).

Gas chromatograms (GC) were measured within the temperature range of 60 to 320° C. with a heating rate of 15° C./min and a run time of 10 min at 320° C. The injector temperature was 250° C. A Zebron ZB-5 column was used (L=30 m, ID=0.25 mm, dj=0.5 μm) with a gas flow rate of 1.5 ml/min. Detection was by flame ionization (FID).

Infrared spectra (FT-IR) were measured as undiluted films on a Nicolet iS5 FT-IR instrument from Thermo Scientific equipped with a horizontal ATR measurement unit with a diamond crystal. Absorption bands are reported in wavenumbers (cm⁻¹).

¹H NMR spectra were measured on a spectrometer of the Bruker Ascend 400 type at 400.14 MHz; the chemical shifts 6 are reported in ppm relative to tetramethylsilane (TMS). No distinction was made between true coupling and pseudo-coupling patterns.

### Preparation of Amines of the Formula (I)

Amine A1: N-(3-Dimethylaminopropyl)-1,3-bis(aminomethyl)benzene 39.2 g (0.30 mol) of 3-formylbenzonitrile (=3-cyanobenzaldehyde) was dissolved under a nitrogen atmosphere at 40° C. in 750 ml of isopropyl alcohol, then 32.0 g (0.315 mol) of 3-dimethylaminopropylamine (DMAPA, from Arkema) was added gradually while stirring, and then the mixture was stirred for 1 hour. Thereafter, the reaction mixture was hydrogenated at 65° C., hydrogen pressure 75 bar, and a flow rate of 5 ml/min on a continuous hydrogenation apparatus with a fixed-bed Raney nickel catalyst, and the hydrogenated solution was concentrated on a rotary evaporator at 65° C., removing unreacted 3-dimethylaminopropylamine, water and isopropanol. 59.9 g of a yellowish oil having a viscosity at 20° C. of 42 mPa·s was obtained.

49.9 g of the oil obtained was then purified by distillation to obtain, at 117 to 120° C. (vapor temperature) and 0.06 mbar, 27.3 g of distillate as a clear, colorless oil with a viscosity at 20° C. of 28 mPa·s, an amine value of 732 mg KOH/g, and an N-(3-dimethylaminopropyl)-1,3-bis(aminomethyl)benzene content determined by GC of >97% (retention time 13.2 min), and a calculated AHEW of 73.7 g/eq, which was used hereinafter as amine A1.

¹H NMR (CDCl₃): 7.28 (m, 2H, Ar—H), 7.20 (m, 2H, Ar—H), 3.85 (d, 2H, Ar—CH₂—N), 3.79 (s, 2H, Ar—CH₂—N), 2.68 (t, 2H, CH₂—N), 2.32 (t, 2H, CH₂—N(CH₃)), 2.21 (s, 6H, CH₃), 1.75 (br s, 3H, NH), 1.68 (m, 2H, C—CH₂—C) FT-IR: 3274, 3023, 2936, 2855, 2812, 2763, 1607, 1589, 1457, 1443, 1376, 1261, 1153, 1114,1040,837,780,738,698

Amine A2: N-(3-(Dimethylamino)propylamino)propyl-1,3-bis(aminomethyl)benzene

The amine A2 was prepared as described for amine A1, except using 26.2 g (0.20 mol) of 3-formylbenzonitrile (=3-cyanobenzaldehyde), 750 ml of isopropanol and 33.4 g (0.21 mol) of 3-(3-(dimethylamino)propylamino)propylamine (DMAPAPA, from Arkema) in place of 3-dimethylaminopropylamine. 49.8 g of a yellowish oil having a viscosity at 20° C. of 42 mPa·s was obtained, which was purified by distillation.

At 148 to 152° C. (vapor temperature) and 0.06 mbar, 26.7 g of distillate was obtained as a clear, colorless oil with a viscosity at 20° C. of 81 mPa·s, an amine value of 794 mg KOH/g, and an N-(3-(dimethylamino)propylamino)propyl-1,3-bis(aminomethyl)benzene content determined by GC of >97% (retention time 15.5 min), and a calculated AHEW of 69.6 g/eq, which was used hereinafter as amine A2.

¹H NMR (CDCl₃): 7.27 (m, 2H, Ar—H), 7.18 (m, 2H, Ar—H), 3.85 (d, 2H, Ar—CH₂—N), 3.77 (s, 2H, Ar—CH₂—N), 2.69 (m, 6H, CH₂—N), 2.29 (t, 2H, CH₂—N(CH₃)), 2.19 (s, 6H, CH₃), 1.78 (br s, 4H, NH), 1.65 (m, 4H, C—CH₂—C) FT-IR: 3294, 3050, 3024, 2912, 2813, 1605, 1588, 1451, 1355, 1336, 1154, 1111, 1027, 890, 779, 732, 696

### Further Substances and Abbreviations Used

| | |
|---|---|
| Araldite ® | bisphenol A diglycidyl ether, |
| GY 250: | EEW 187 g/eq (from Huntsman) |
| Araldite ® | monoglycidyl ethers of C₁₂ to C₁₄ |
| DY-E: | alcohols, EEW about 290 g/eq (from Huntsman) |
| DMAPAPA: | 3-(3-(dimethylamino)propylamino)propylamine, |
| | AHEW 53 g/eq (DMAPAPA, from Arkema) |
| B-EDA | N-benzylethane-1,2-diamine, prepared as described below, |
| | 150.2 g/mol, AHEW 50.1 g/eq |
| IPDA: | 1-amino-3-aminomethyl-3,5,5-trimethylcyclohexane, |
| | AHEW 42.6 g/eq (Vestamin ® IPD from Evonik) |
| Ancamine ® | 2,4,6-tris(dimethylaminomethyl)phenol (from Evonik) |
| K54 | |

N-Benzylethane-1,2-diamine (B-EDA):

An initial charge of 180.3 g (3 mol) of ethane-1,2-diamine at room temperature was mixed with a solution of 106.0 g (1 mol) of benzaldehyde in 1200 ml of isopropanol and stirred for 2 hours, then hydrogenated at 80° C., hydrogen pressure 80 bar, and a flow rate of 5 ml/min in a continuous hydrogenation apparatus with a fixed-bed Pd/C catalyst, and the hydrogenated solution was concentrated on a rotary evaporator at 65° C., removing unreacted ethane-1,2-diamine, water and isopropanol. The resulting reaction mixture was purified by distillation at 80° C. under reduced pressure. This gave a colorless liquid having an N-benzylethane-1,2-diamine content determined by GC of >97%.

### Preparation of Curing Agents and Epoxy Resin Compositions:

### Examples 1 and 2

For each example, the ingredients of the resin component specified in table 1 were mixed in the specified amounts (in parts by weight) using a centrifugal mixer (SpeedMixer™ DAC 150, FlackTek Inc.) and stored with exclusion of moisture. The ingredients of the curing agent component specified in table 1 were processed and stored in a similar manner.

The two components of each composition were then processed using the centrifugal mixer into a homogeneous liquid and this was tested immediately as follows:

Viscosity was measured in the described manner at a temperature of 20° C. 5 min after mixing the resin component and curing agent component.

Shore D hardness was determined in accordance with DIN 53505 on two cylindrical test specimens (diameter 20 mm, thickness 5 mm), one of which was stored under standard climatic conditions and the other at 8° C. and 80% relative humidity, and the hardness measured in each case after 1 day (24 h) and after 2 days.

In addition, a film was applied to a glass plate in a layer thickness of 500 m, and this was stored/cured under standard climatic conditions. König's hardness (König's pendulum hardness to DIN EN ISO 1522) was determined on this film after 1 day, 2 days, 7 days and 14 days (1d SCC), (2d SCC), (7d SCC), (14d SCC). After 14 days, the appearance (SCC) of the film was assessed. A clear film was described as “nice” if it had a glossy and nontacky surface with no structure. “Structure” refers to any kind of marking or pattern on the surface.

The results are reported in Table 1.

TABLE 1
Composition and properties of examples 1 and 2

| | | |
|---|---|---|
| Example | 1 | 2 |

| Resin component: | | | | |
|---|---|---|---|---|
| Araldite ® GY 250 | | | 167.2 | 167.2 |
| Araldite ® DY-E | | | 31.8 | 31.8 |
| Curing agent component: | | | | |
| Amine A1 | | | 73.7 | — |
| Amine A2 | | | — | 69.6 |
| Viscosity (10′) [Pa · s] | | | 0.41 | 0.46 |
| | Shore D | (1 d SCC) | 67 | 51 |
| | | (2 d SCC) | 67 | 57 |
| | Shore D | (1 d 8°/80%) | 45 | n.m.¹ |
| | | (2 d 8°/80%) | 47 | 46 |
| | König | (1 d SCC) | 146 | 140 |
| | hardness | (2 d SCC) | 155 | 143 |
| | [s] | (7 d SCC) | 157 | 143 |
| | | (14 d SCC) | 157 | 144 |

| | | |
|---|---|---|
| Appearance (SCC) | nice | nice |

¹not measurable (too soft)

### Examples 3 to 6

For each example, the ingredients of the resin and of the curing agent component specified in table 2 were mixed as described for example 1, and the mixed components were tested as follows:

Viscosity, Shore D, Konig hardness (SCC) and Appearance (SCC) were determined as described for example 1.

Gel time was determined by moving a freshly mixed amount of about 3 g under standard climatic conditions with a spatula at regular intervals until the mass underwent gelation.

In addition, a further film was applied to a glass plate in a layer thickness of 500 μm and this was immediately after application stored/cured for 7 days at 8° C. and 80% relative humidity and then for 2 weeks under standard climatic conditions. 24 hours after application, a polypropylene bottle top beneath which a damp sponge had been positioned was placed on the film. After a further 24 hours, the sponge and the bottle top were removed and positioned at a new point on the film, from which it was in turn removed and repositioned after 24 hours, this being done a total of 4 times. The appearance of this film was then assessed (designated “Appearance (8°/80%)” in the tables) in the same way as described for Appearance (SCC). Also reported in each case here was the number and nature of visible marks that had formed in the film as a result of the damp sponge or the bottle top on top. The number of white discolored spots was reported as “blushing”. A faint white discolored spot was designated as “(1)”. A clear white discolored spot was designated as “(1)”. The designation “ring” was reported if a ring-shaped imprint was present due to sinking of the first bottle top applied 24 hours after application. Such a ring-shaped impression indicates that the coating was not ready to be walked on. A very minimal ring-shaped imprint was designated as “(yes)”. A clear ring-shaped imprint was designated as “yes”. König's hardness was again determined on the films thus cured, in each case after 7 days at 8° C. and 80% relative humidity (König hardness (7d 8°/80%)) and then after a further 2 days under SCC (König hardness (+2d SCC)), 7 days under SCC (Konig hardness (+7d SCC)), and 14 d under SCC (Konig hardness (+14d SCC)).

The results are reported in Table 2.

Comparative examples are labelled “(Ref.)”.

TABLE 2
Composition and properties of examples 3 to 6.

| Example | | 3 | 4 | 5 (Ref.) | 6 (Ref.) |
|---|---|---|---|---|---|
| Resin component: | | | | | |
| Araldite ® GY 250 | | 167.2 | 167.2 | 167.2 | 167.2 |
| Araldite ® DY-E | | 31.8 | 31.8 | 31.8 | 31.8 |
| Curing agent component: | | | | | |
| Amine A1 | | 22.1 | 11.1 | — | — |
| DMAPAPA | | — | — | 8.0 | — |
| B-EDA | | — | 7.5 | 7.5 | 15.0 |
| IPDA | | 29.8 | 29.8 | 29.8 | 29.8 |
| Benzyl alcohol | | 30.0 | 30.0 | 30.0 | 30.0 |
| Salicylic acid | | 2.0 | 2.0 | 2.0 | 2.0 |
| Ancamine ® K54 | | — | — | — | 2.0 |
| Viscosity (10′) [Pa · s] | | 0.54 | 0.49 | 0.43 | 0.46 |
| Gel time (h:min) | | 3:30 | 4:00 | 4:00 | 4:00 |
| Shore D | (1 d SCC) | 77 | 71 | 67 | 57 |
| | (2 d SCC) | 79 | 76 | 73 | 74 |
| Shore D | (1 d 8°/80%) | 33 | 13 | 11 | 9 |
| | (2 d 8°/80%) | 76 | 69 | 63 | 61 |
| König | (1 d SCC) | 105 | 49 | 53 | 34 |
| hardness | (2 d SCC) | 157 | 119 | 118 | 85 |
| [s] | (7 d SCC) | 183 | 168 | 163 | 146 |
| | (14 d SCC) | 185 | 181 | 172 | 158 |
| Appearance (SCC) | | nice | nice | nice | nice |
| König | (7 d 8°/80%) | 105 | 59 | 49 | 32 |
| hardness | (+2 d SCC) | 164 | 153 | 131 | 106 |
| [s] | (+7 d SCC) | 175 | 167 | 147 | 137 |
| | (+14 d SCC) | 183 | 160 | 143 | 137 |

| | | | | |
|---|---|---|---|---|
| Appearance (8°/80%) | nice | nice | haze | nice |
| Blushing | (1) | (1) | 1 | (1) |
| Ring | (yes) | (yes) | yes | yes |

1. A method for curing of epoxy resins, comprising contacting an epoxy resin with an amine of the formula (I): 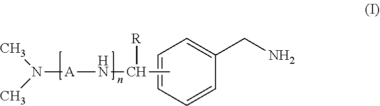
where n is 0 or 1 or 2, R is H or methyl and A is an alkylene radical having 2 to 6 carbon atoms.

2. The method as claimed in claim 1, wherein R is H.

3. The method as claimed in claim 1, wherein A is 1,2-ethylene or 1,3-propylene.

4. The method as claimed in claim 1, wherein n is 0 or 1.

5. The method as claimed in claim 1, wherein the amine of the formula (I) is selected from the group consisting of N,N-dimethyl-1,3-bis(aminomethyl)benzene, N-(2-dimethylaminoethyl)-1,3-bis(aminomethyl)benzene, N-(3-dimethylaminopropyl)-1,3-bis(aminomethyl)benzene, N,N-dimethyl-1,4-bis(aminomethyl)benzene, N-(2-dimethylaminoethyl)-1,4-bis(aminomethyl)benzene, N-(3-dimethylaminopropyl)-1,4-bis(aminomethyl)benzene, N,N-dimethyl-1,2-bis(aminomethyl)benzene, N-(2-dimethylaminoethyl)-1,2-bis(aminomethyl)benzene and N-(3-dimethylaminopropyl)-1,2-bis(aminomethyl)benzene.

6. The method as claimed in claim 1, wherein the amine of the formula (I) is a constituent of a reaction product obtained from the reductive alkylation of at least one amine of the formula (II) with at least one benzonitrile of the formula (III) and hydrogen.

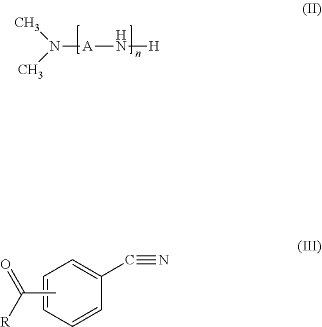

7. A curing agent for epoxy resins comprising at least one amine of the formula (I) as claimed in claim 1 and at least one further constituent selected from further amines A1, accelerators, and thinners.

8. The curing agent as claimed in claim 7, wherein at least one further amine A1 is present, where the further amine A1 has at least three aliphatic amine hydrogens.

9. An epoxy resin composition comprising
a resin component comprising at least one epoxy resin and
a curing agent component comprising at least one amine of the formula (I) as claimed in claim 1 or a curing agent for epoxy resins comprising at least one amine of the formula (I) and at least one further constituent selected from further amines A1, accelerators, and thinners.

10. A cured composition obtained from the epoxy resin composition as claimed in claim 9 after the resin component and the curing agent component have been mixed.

11. A method for coating, comprising the steps of
(i) applying the mixed epoxy resin composition as claimed in claim 9 to at least one substrate,
(ii) optionally applying a further layer of the mixed epoxy resin coating as claimed in claim 9 to the cured layer.

12. A method of bonding, comprising the applying of the mixed epoxy resin composition as claimed in claim 9
either to at least one of the substrates to be bonded and joining the substrates to form a bond,
or into a cavity or gap between two or more substrates and optionally inserting an anchor into the cavity or gap.

13. A method of producing a shaped body, comprising the introducing of the mixed epoxy resin composition as claimed in claim 9 into a casting mold, followed by the curing of the mixed composition, optionally under pressure and optionally by means of supplied heat.

14. An amine of the formula (Ia) 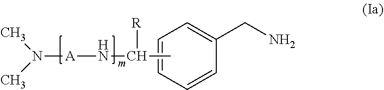
where
m is 1 or 2,
R is H or methyl, and
A is an alkylene radical having 2 to 6 carbon atoms.

15. A reaction product comprising at least one amine of the formula (Ia), obtained from the reductive alkylation of at least one amine of the formula (IIa) with at least one benzonitrile of the formula (III) and hydrogen, 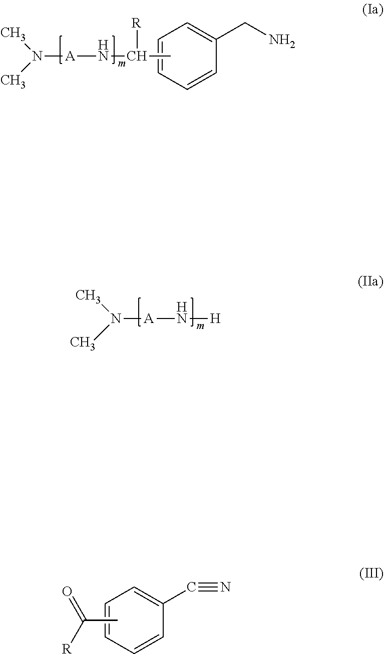
where
m is 1 or 2,
R is H or methyl, and
A is an alkylene radical having 2 to 6 carbon atoms.
